(19) United States
(12) Patent Application Publication
(10) Pub. No.: US 2005/0158423 A1
(43) Pub. Date: Jul. 21, 2005

(54) Method of improving food fermentation procedures

(76) Inventors: Arnold Geis, Nortorf (DE); Andrea Budde-Niekiel, Burg (DE); Mohamed El-Demerdash Hassan, Ismallia (EG); Knut J. Heller, Kiel (DE)

Correspondence Address: Steptoe & Johnson, 1330 Connecticut Ave N W, Washington, DC, 20036, US

(21) Appl. No.: 10/506,214

(22) PCT Filed: Mar. 11, 2003
(86) PCT No.: PCT/IB03/01429

(30) Foreign Application Priority Data
Mar. 11, 2002 (GB) 0205647.1

(51) Int. Cl. A23C 9/12; C12N 1/21

(52) U.S. Cl. 426/43; 435/252.3

(57) ABSTRACT

The present invention relates to the field of food fermentation procedures involving lactic acid bacteria and in particular to improving the quality of dairy products like acidity, post-fermentation acidification, aroma, flavor, mildness, consistency and texture. Specifically, the present invention provides a method for making dairy products using the thermophilic Streptococcus thermophilus and the mesophilic Lactococcus lactis carrying plasmids encoding small heat shock proteins which allow fermentation to be carried out at temperatures higher than the regular fermentation temperatures. The thermophilic and mesophilic lactic acid bacterial species expressing said small heat shock proteins also exhibit increased tolerance to lower pH and higher salt concentrations as well as reduced sensitivity to bacteriophage attack at the elevated fermentation temperatures.

### FIELD OF THE INVENTION

The present invention relates to the field of food fermentation procedures involving lactic acid bacteria and in particular to improving the quality of dairy products like acidity, post-fermentation acidification, aroma, flavour, mildness, consistency and texture. Specifically, the present invention provides a method for making dairy products using the thermophilic Streptococcus thermophilus and the mesophilic Lactococcus lactis expressing small heat shock protein which allows fermentation to be carried out at temperatures higher than the regular fermentation temperatures. The thermophilic and mesophilic lactic acid bacterial species expressing said heat shock proteins also exhibit increased tolerance to lower pH and higher salt concentrations as well as reduced sensitivity to bacteriophage attack at the elevated fermentation temperatures.

### INTRODUCTION

In the food industry, the homofermentative, thermophilic lactic acid bacterium Streptococcus thermophilus is used as a starter species in dairy fermentations mostly in combination with thermophilic lactobacilli. Commercially, the most important of such fermentations is yoghurt production. Yoghurt results from the protosymbiotic growth of the two lactic acid bacteria Streptococcus thermophilus and Lactobacillus delbrueckii subsp. bulgaricus. Protosymbiosis characterises the metabolic interrelationships between organisms which in this respect means that both lactic acid bacteria promote each other's growth by providing nutrients required for optimal growth. As a consequence, acidification is rapid and results in final pH values as low as 3.5.

Although it is generally accepted that Lactobacillus delbrueckii subsp. bulgaricus is the yoghurt starter component mostly responsible for flavour development, results obtained from fermentation experiments using different combinations of Streptococcus thermophilus and Lactobacillus delbrueckii subsp. bulgaricus strains indicate a considerable impact of Streptococcus thermophilus on flavour development (C. Möller, W. Bockelmann, and K. J. Heller, unpublished).

The number of consumers preferring yoghurt with milder aroma has been increasing during recent years. Such milder taste may be achieved if fermentation stops already at pH values between 4.0 and 4.7. In addition, post-fermentation acidification is a major cause of bitterness and/or sourness in yoghurt. In order to overcome this undesirable characteristic of yoghurt, the development of bitterness and/or sourness can be inhibited by using starter combinations showing no or only little post-fermentation acidification.

In addition to yoghurt, thermophilic starter cultures are also used for production of different types of hard cheese, as well as semi-hard cheese like mozzarella, and acid curd cheese. Especially for the latter cheese the final pH obtained after fermentation has a considerable impact on the quality of the product.

The mesophilic lactic acid bacterium Lactococcus lactis is used as a starter component for many cheese varieties and butter milk. While with cheese production usually acidification is terminated well above pH 5.0 by cutting the curd and concomitant removal of the fermentable lactose, butter milk fermentation is terminated at a pH of 4.6, which is very close to the lowest pH reached by this organism.

In warm countries, like e.g. Egypt, keeping-time of raw milk is very short, due to unsatisfactory cooling systems. To overcome the problem of rapid spoilage, NaCl is added to the milk in relatively high quantities. This, on the other hand, interferes with subsequent dairy fermentations. Therefore, there is a strong demand for salt-tolerant starter cultures in such countries.

Dairy fermentations are carried out by starter organisms with different temperature requirements. Fermentations involving starter organisms with different temperature requirements at one temperature lead to sub optimal growth of one of the organisms. By way of example, in the case of yoghurt-production, cell count numbers of Lactobacillus delbrueskii ssp. bulgaricus in the final product generally are too low due to fermentation at sub optimal temperatures.

Thus there is a pressing need to develop lactic acid bacteria which can ferment milk at higher temperatures and salt concentrations than their regular fermentation temperatures and salt concentrations and species which remain viable for longer at lower pH values. On the other hand, fermentation at higher temperature would suppress the raise of contaminants. For this reason the salt concentration might be reduced with benefit for human health.

Aspects of the present invention are presented in the accompanying claims and in the following description and drawings. These aspects are presented under separate section headings. However, it is to be understood that the teachings under each section are not necessarily limited to that particular section heading.

### SUMMARY OF THE INVENTION.

Intriguingly, the inventors of the present patent application show that certain species of lactic acid bacteria which express small heat shock proteins can ferment milk at temperatures and/or salt concentrations higher than the normal milk fermentation temperatures and/or salt concentrations. The inventors of the present patent application further show that lactic acid bacteria which express heat shock proteins remain viable for longer at lower pH conditions and due to the high fermentation temperature become less sensitive to bacteriophage attack.

Importantly, the inventors have found that the lactic acid bacteria of the present invention when incubated with milk can produce a fermentation product with milder taste characteristics when compared to the fermentation product produced by corresponding lactic acid bacteria which do not express heat shock proteins.

Accordingly, the invention pertains in a first aspect to a method of preparing mild fermentation based dairy products, said method comprising the steps of: combining milk with a lactic acid bacterium capable of expressing a small heat shock protein, and culturing the milk/bacterium mixture at a temperature higher than a regular fermentation temperature and/or at a salt concentration higher than a regular fermentation salt concentration wherein the fermentation product has milder taste characteristics when compared to the fermentation product produced by the corresponding lactic acid bacteria which do not express small heat shock proteins.

In another aspect, there are provided thermophilic and/or mesophilic lactic acid bacteria which are used in dairy fermentation processes, which carry genes coding for small heat shock proteins and which produce a fermentation product with milder taste characteristics when compared to the fermentation product produced by the corresponding thermophilic and/or mesophilic lactic acid bacteria which do not express the small heat shock proteins of the present invention.

Preferably, the pH of the product is above 4.0.

In another aspect the present invention provides a method comprising combining milk with a first and a second lactic acid bacterium, in which each at least one of the first and/or second lactic acid bacteria is capable of expressing a small heat shock protein, and culturing the milk/bacterium mixture as specified.

In another aspect, the present invention also provides a method in which each of the first lactic acid bacterium and the second lactic acid bacterium comprises a mesophilic bacterium.

In another aspect, the present invention also provides a method in which each of the first lactic acid bacterium and the second lactic acid bacterium comprises a thermophilic bacterium.

In another aspect, the present invention also provides a method in which the milk is combined with a starter culture comprising a first thermophilic lactic acid bacterium prior to the addition of a second thermophilic lactic acid bacterium.

In another aspect, the present invention also provides a method in which the first lactic acid bacterium comprises a mesophilic lactic acid bacterium and the second lactic acid bacterium comprises a thermophilic lactic acid bacterium.

In another aspect, the present invention also provides a method in which the milk is combined with a starter culture comprising a mesophilic lactic acid bacterium prior to the addition of a thermophilic lactic acid bacterium.

In another aspect, the present invention also provides a method where each mesophilic bacterium is independently selected from a group consisting of but not limited to: Lactococcus spp, Leuconostoc spp, Lactococcus lactis, Lactococcus lactis subspecies cremoris, and Lactacoccus lactis subspecies lactis.

In another aspect, the present invention also provides a method where each thermophilic bacterium is independently selected from a group consisting of but not limited to: Steptococcus spp, Bifidobacterium spp, Pediococcus spp, Lactobacillus spp, Steptococcus thermophilus, Lactobacillus delbrueckii or Lactobacillus delbrueckii subspecies bulgaricus containing plasmids which carry genes coding for small heat shock proteins.

In yet another aspect, there are provided mesophilic and thermophilic lactic acid bacteria which have increased acid tolerance.

In a further aspect, there are provided mesophilic and thermophilic lactic acid bacterial species which have decreased sensitivity to bacteriophage attack as a result of the elevated fermentation temperature.

In yet a further aspect, the invention relates to milk that is obtained by the method of the present invention and a fermentation product with milder taste characteristics that is obtained by fermenting such a milk, such as yoghurt, and acid curd cheese, and hard cheese, and semi-hard cheese like mozzarella, and fresh cheese, and quark, and butter milk, and cottage cheese, when compared to the fermentation product produced by the corresponding lactic acid bacteria which do not express the small heat shock proteins of the present invention.

In yet another aspect, the invention relates to milk that is obtained by the method of the present invention and a fermentation product that is obtained by fermenting such as milk wherein the fermentation product can be selected from a group consisting of acid curd cheese, hard cheese, semi-hard cheese like mozzarella, fresh cheese, quark, butter milk, Swiss type cheese or cottage cheese.

In yet a further aspect, the invention relates to milk that is obtained by the method of the present invention and milder taste characteristics yoghurt that is obtained by fermenting such a milk when compared to the yoghurt produced by the corresponding lactic acid bacteria which do not express the small heat shock proteins of the present invention.

In another aspect, the present invention provides a method of fermenting milk in which the lactic acid bacterium comprises a recombinant lactic acid bacterium, which has been engineered to express a small heat shock protein at a higher level than a corresponding unmodified bacterium.

In another aspect, the present invention provides a method of fermenting milk in which the lactic acid bacterium comprises a recombinant lactic acid bacterium, which has been engineered to over-express a small heat shock protein when compared to a corresponding unmodified bacterium.

In another aspect, there are provided thermphilic and/or mesophilic lactic acid bacteria which are used in dairy fermentation processes, which carry genes for small heat shock proteins.

In another aspect, the present invention provides a recombinant microorganism which can carry out milk fermentation at temperatures and/or salt concentrations higher than regular fermentation and/or salt concentrations.

It is known that small heat shock protein(s) (shsp) form one of the families of molecular chaperones preventing the irreversible aggregation and assisting in the refolding of denatured proteins. These proteins exist in all organisms and are characterized by a low molecular mass in the range of 12-30 kDa, oligomeric structure and the presence of a conserved domain. This domain is highly similar to α-cristallins of vertebrates. It is preferred that the small heat shock proteins which are capable of being used in the methods of the present invention have a molecular mass in the range of 16-18 kDa.

Typically the term “small heat shock protein(s) or (sHsp)” refers to an ATP-independent sHsp capable of binding and stabilizing denatured proteins and preventing their irreversible aggregation. Proteins stabilized in this way can be subsequently refolded by an ATP-dependent chaperone system.

Analysis of complete bacterial genome sequences revealed, that most bacteria have only a small number of small heat shock proteins. In the lactic acid bacteria only a few strains of S. thermophilus carry genes for sHsps, which are located on small plasmids separate from the bacterial genome.

Although the present invention is described with reference to small heat shock proteins, it should be understood by those of skill in the art that other types of heat shock proteins can also be adapted and used in the method of the present invention.

### DETAILED DESCRIPTION OF THE PRESENT INVENTION

Improved Lactic Acid Fermentation.

A primary objective of the present invention is to provide a method to improve the properties of fermentation based milk products. This is, in respect of yoghurt, achieved by providing a method for preparing yoghurt which has highly desirable characteristics with respect to milder aroma, acidity, post-fermentation acidification, taste and appearance and which additionally has an extended shelf life as reflected in the reduced whey production, the mildness of the aroma and higher density of live cultures i.e. the staling of the fermented milk product is retarded relative to a milk product made without use of the microorganisms of the present invention.

As used herein, the term “milder taste characteristics” refers to fermented milk products characterised with significantly reduced bitterness and/or sourness. In addition or in the alternative, the term “milder taste characteristics” may also be interpreted with respect to the pH of the product. Accordingly, the product of the present invention which has a mild taste characteristics has a pH above 4.0, preferably in the range 4.0 to 5.0, even more preferably 4.0 to 4.7. Accordingly, milder taste characteristics fermentation milk products refer to the fermentation milk products obtained by incubating the lactic acid bacteria of the present invention with milk according to the method of the present invention.

As used herein the term “corresponding lactic acid bacteria which do not express the small heat shock proteins of the present invention” refers to lactic acid bacteria which have been cured of (i.e. they have lost) the plasmid which carries the small heat shock proteins of the present invention (see Examples). The curing of a bacteria from a plasmid can be achieved by methods known in the art. Alternatively, the lactic acid bacteria which do not express the heat shock proteins of the present invention may still contain the plasmid but the genes coding for the shsp may be specifically modified using genetic recombination and/or mutagenesis techniques to prevent the expression of the genes.

Although it is presently preferred to use the method of the invention for yoghurt production, the use of the method for any other type of fermentation based milk product such as hard cheese, semi-hard cheese like mozzarella, acid curd cheese, quark, butter milk, fresh cheese and cottage cheese the quality of which can be improved by the addition of the microorganisms of the present invention, is also contemplated.

The milk in respect of the present invention can be obtained from a ruminant animal, preferably selected from a group consisting of buffalo, cow, sheep, lama, goat or camel. The milk can also be skimmed or semi-skimmed. The milk may also be derived from plants such as soy or rice or it can be synthetically generated milk.

Synthetically generated milk is a term used in the art to describe an artificial product which is similar to milk. The composition of the synthetic milk may be specifically designed to exclude for example certain components to which consumers may be intolerant or allergic. The synthetic milk can also be a fortified milk. By way of example, the fortified milk may contain additional compounds which may or may not have nutritional value such as minerals, vitamins, polysaccharides, oligosaccharides, thickeners or water absorbing materials.

Generally, temperature range for growth of an organism is a characteristic of considerable taxonomical value. Depending on their preferred growth temperature, bacteria in general and that includes lactic acid bacteria are divided broadly into mesophilic and thermophilic bacteria. It is known that mesophilic bacteria have an optimum temperature of between 20° C. to 44° C. While the optimum temperature of thermophilic bacteria is in the range of between 35° C. to 55°.

The present invention relates to methods for producing different milk products by fermenting milk with combinations of mesophic and/or thermophilic lactic acid bacteria.

According to the present invention, mesophilic lactic acid bacteria are defined as bacteria that have a regular growth temperature in the range of between 20°-30° C. However, mesophilic lactic acid bacteria which exhibit a regular growth temperature of between 30° C. and 44° C. may also be used in the methods of the present invention.

According to the present invention, thermophilic lactic acid bacteria are defined as bacteria that have a regular growth temperature in the range of between 35° C.-47° C. However, thermophilic lactic acid bacteria which exhibit a regular growth temperature of between 50° C. to 55° may also be used in the methods of the present invention.

The summary below lists a number of different mesophilic and thermophilic lactic acid bacteria which is for the purposes of illustration and not in any way limited to the specific lactic acid bacteria included in the summary below.

Optimal growth temperature of starter cultures

| Classification of | | Range of optimal |
|---|---|---|
| culture | Species | growth temperature |
| mesophilic culture | Lactococcus lactis | 18-30° C. |
| | Leuconostoc | 18-30° C. |
| thermophilic culture | Streptococcus | 35-42° C. |
| | thermophilus | |
| | Lactobacillus del. | 43-46° C. |
| | bulgaricus | |

The present method comprises as an essential step that an effective amount of lactic acid bacteria of the invention which under fermentation conditions are capable of fermenting milk at a higher temperature than regular fermentation temperatures: up to 43° C. for the mesophilic lactococci (especially for Lactococcus lactis subsp. cremoris) and up to 55° C. for the thermophilic Streptococcus thermophilus.

The fermentation temperature of yoghourt is 42° C. (or between 40°-43° C.), which is a compromise between the optima of the two starter organisms Streptococcu thermophilus (39° C.) and Lactobacillus delbrueckii subsp. Bulgaricus (45° C.). When the Streptococcus thermophilus carries the shsp the optimum fermentation temperature is altered and the optimum temperature of he organsim is increased. This means that you can ferment the yoghourt at 45° C. and then both Lactobacillus delbrueckii subsp. Bulgaricus and Streptococcus thermophilis carrying shsp will grow at optimum temperatures.

Accordingly, the present invention provides mesophilic lactic acid bacteria which as a result of expressing small heat shock proteins according to the present invention are capable of fermenting milk at temperatures which are higher than the corresponding mesophilic lactic acid bacteria which do not express small heat shock proteins according to the present invention.

The present invention also provides the thermophilic lactic acid bacteria Streptococcus thermophilus which when expressing the small heat shock proteins according to the present invention is capable of fermenting milk in the range of 42° C. to 55° C. which is higher than the optimum temperature for the corresponding Streptococcus thermophilus which do not express small heat shock proteins according to the present invention.

Importantly, an effective amount of small heat shock protein expressing Streptococcus thermophilus bacteria when incubated with an effective amount of Lactobacillus del. bulgaricus can ferment milk at higher than normal temperatures, which would lead to the production of a milder taste characteristics product.

As used herein, the term “effective amount” describes an amount of thermophilic and/or mesophilic bacterial species such as Streptococcus thermophilus or Lactococcus lactis respectively which is sufficient to ferment detectable amount of sugar compounds present in the milk. More specifically, presence of the microorganisms of the present invention may not only result in detectable fermentation of sugar compounds in milk but which in addition results in the formation of fermentation end products at a level which result in improved properties of the milk products, such as significantly improved acidity, texture, taste, appearance and the mildness of the aroma which can be ascribed to the addition of the microorganisms of the present invention.

In one aspect, the invention provides Streptococcus thermophilus which ferment milk at elevated temperatures for the production of mild aroma fermented milk products which comprise but are not limited to yoghurt, acid curd cheese, hard cheese, semi- hard cheese like mozzarella, or fresh cheese.

In another aspect, the present invention provides Lactococcus lactis which ferment milk at elevated temperatures for the production of mild aroma fermented milk products which comprise but are not limited to quark, butter milk, fresh cheese, or cottage cheese.

As used herein the terms “elevated temperatures” or “higher temperatures” are used interchangeably and refer to the ability of mesophilic lactic acid bacteria and thermophilic lactic acid bacteria to ferment milk at higher than normal fermentation temperatures.

For mesophilic lactic acid bacteria the term “elevated temperatures” refer to the fermentation of milk at between 39° C.-48° C., preferably between 41° C-47° C., more preferably between 43° C.-46° C., even more preferably between 43° C.-46° C. and most preferably at 43° C. compared to the regular fermentation temperature range (see Summary above)

In the case of thermophilic lactic acid bacteria the term “elevated temperatures” refers to the fermentation of milk at between 48° C.-58° C. preferably between 53° C.-57° C., more preferably between 54° C.-57° C., even more preferably between 54° C.-56° C. and most preferably at 55° C. compared to the regular fermentation temperature range (see Summary above).

In addition, the availability of mesophilic starter bacteria growing at temperatures above 39° C. allows new combinations of mesophilic and/or thermophilic starters for production of new fermented products.

In yet another aspect, the present invention provides lactic acid bacteria with reduced bacteriophage attack sensitivity. This is as a result of the ability of the lactic acid bacteria according to the present invention to ferment milk at elevated temperatures.

In another aspect, the present invention provides lactic acid bacteria with increased tolerance to higher concentrations of salt for example higher than 1%, preferably higher than 2%, and more preferably higher than 2.5%. It is of note that the normal salt concentration of milk is in the range of 0.7 to 0.8%.

This feature of the microorganisms of the present invention are particularly important for southern countries where salt is added to milk to inhibit growth of spoilage and pathogenic microorganisms. Such milk may be more easily fermented by the lactic acid bacteria of the present invention when producing milder taste characteristics milk product obtained by fermenting such a milk when compared to the milk product produced by the corresponding lactic acid bacteria which do not express the small heat shock proteins of the present invention.

In another aspect, the present invention provides a fermentation product comprising lactic acid bacteria expressing small heat proteins of the present invention with increased shelf life of the product when stored at 4° C.

In another aspect, the present invention provides the product of yoghurt with thermophilic starter cultures above 42° C. with higher cell count numbers of viable Lactobacillus sp. in the final product.

In another aspect, the present invention provides a method of fermenting milk in which the lactic acid bacterium comprises a recombinant lactic acid bacterium, which has been engineered to express a small heat shock protein at a higher level than a corresponding unmodified bacterium which can carry out milk fermentation at temperatures and/or salt concentrations higher than regular fermentation and/or salt concentrations.

In another aspect, the present invention provides a method of fermenting milk in which the lactic acid bacterium comprises a recombinant lactic acid bacterium, which has been engineered to over-express a small heat shock protein when compared to the corresponding unmodified bacterium which can carry out milk fermentation at temperatures and/or salt concentrations higher than regular fermentation and/or salt concentrations.

In one aspect, the present invention provides polypeptides derived from pSt04 and pER1-1.

In one aspect, the present invention provides polypeptides consisting essentially of the amino acid sequences presented as SEQ ID No: 1 and SEQ ID No: 2 for use in the method of the present invention.

As used herein the term “amino acid sequence” refers to peptide, polypeptide or protein sequences or variant, homologue, fragment or derivative thereof

The term “derivative” as used herein includes chemical modifications of the polypeptide that can be used in the method of the present invention. Illustrative of such modifications would be replacement of hydrogen by an alkyl, acyl or amino group.

Preferably, the polypeptide that can be used in the method of the present invention does not cover the native polypeptide when it is in its natural environment and when it has been expressed by its native nucleotide coding sequence which is also in its natural environment and when that nucleotide sequence is under the control of its native promoter which is also in its natural environment.

More preferably, the polypeptide that can be used in the method of the present invention covers the native polypeptide when it is in its natural environment and when it has been expressed by its native nucleotide coding sequence which is also in its natural environment and when that nucleotide sequence is under the control of its native promoter which is also in its natural environment.

In one aspect of the present invention, the polypeptides will provide improved methods of fermentation of milk by lactic acid bacteria in the production of milder taste characteristics yoghurt when compared to the yoghurt produced by the corresponding lactic acid bacteria which do not express the polypeptides of the present invention.

The present invention also covers the nucleotide sequence(s) essentially presented as SEQ ID No: 3 and SEQ ID No: 4 coding for polypeptides that can be use in the method of the present invention.

In the context of the present invention, the nucleotide sequence coding for the polypeptides that can be used in the method of the present invention may be the same as the naturally occurring form, or is a variant, homologue, fragment or derivative thereof. Preferably, the nucleotide sequence coding for the polypeptide that can be used in the method of the present invention is contained within a lactic acid bacteria.

The term “nucleotide sequence” as used herein refers to an oligonucleotide sequence or polynucleotide sequence, and variants, homologues, fragments and derivatives thereof (such as portions thereof).

The nucleotide sequence may be DNA or RNA of genomic or synthetic or recombinant origin which can be double-stranded or single-stranded whether representing the sense or antisense strand. The nucleotide sequence need not necessarily be a complete naturally occurring DNA sequence. Thus, the DNA sequence can be, for example, a synthetic DNA sequence, a recombinant DNA sequence (i.e. prepared by use of recombinant DNA techniques), a cDNA sequence or a partial genomic DNA sequence, including combinations thereof. The DNA sequence need not be a coding region. If it is a coding region, it need not be an entire coding region. In addition, the DNA sequence can be in a sense orientation or in an anti-sense orientation. Preferably, it is in a sense orientation. However, in some instances, where the anti-sense DNA sequence is transcribed and used as a template for protein synthesis, the anti-sense sequence is preferred. Preferably, the DNA is or comprises a cDNA.

Thus, preferably the term “nucleotide sequence” means DNA. More preferably, the term “nucleotide sequence” means DNA prepared by use of recombinant DNA techniques (i.e. recombinant DNA). Thus, preferably, the present invention relates to a DNA sequence (preferably a cDNA sequence) encoding the polypeptides that can be used in the method of the present invention.

In one preferred aspect, the present invention provides a nucleotide sequence consisting essentially of the polypeptide that can be used in the method of the present invention coding sequence.

In another aspect, the present invention provides a nucleotide sequence coding for the polypeptide that can be used in the method of the present invention presented in SEQ ID No: 3 and SEQ ID No: 4.

In a further aspect, the present invention provides a nucleotide sequence coding for the polypeptide that can be used in the method of the present invention presented in SEQ ID No: 3 or SEQ ID No: 4 or variant, homologue, fragment or a derivative thereof.

Altered polynucleotide sequences which code for the polypeptide/small heat shock protein that can be used in the method of the present invention may be used in accordance with the invention and may include different nucleotide residues resulting in a polynucleotide that encodes the same or a functionally equivalent polypeptide of the present invention which polypeptide may have deletions, insertions or substitutions therein.

As used herein a “deletion” is defined as a change in either nucleotide or amino acid sequence in which one or more nucleotides or amino acid residues, respectively, are absent.

As used herein an “insertion” or “addition” is a change in a nucleotide or amino acid sequence which has resulted in the addition of one or more nucleotides or amino acid residues, respectively, as compared to the naturally occurring polypeptide of the invention or the encoding nucleotide sequence.

As used herein “substitution” results from the replacement of one or more nucleotides or amino acids by different nucleotides or amino acids, respectively.

In accordance with the present invention, nucleotide sequences coding for the polypeptide, fragments of the polypeptide, fusion proteins or functional equivalents thereof that can be used in the method as described, which may be used to generate recombinant DNA molecules that direct the expression thereof in appropriate host cells. In one embodiment of the present invention, a nucleotide sequence encoding the polypeptide that can be used in the method of the present invention is operably linked to a promoter sequence capable of directing expression of the nucleotide sequence coding for the polypeptide in a suitable host cell. When introduced into the host cell, the transformed host/recombinant microorganism may be cultured under suitable conditions until sufficient levels of the polypeptide of the invention are achieved after which the cells can be used for fermentation of milk under the method of the present invention.

Peptide Sequence

The terms “variant”, “homologue”, “derivative” or “fragment” in relation to the polypeptide sequence that can be used in the method of the present invention, include any substitution of, variation of, modification of, replacement of, deletion of or addition of one (or more) nucleic acid from or to the sequence providing the expression product of the resultant nucleotide sequence would allow lactic acid bacteria to ferment milk at higher than normal temperatures and/or salt concentrations, preferably having at least the same capacity to allow lactic acid bacteria to ferment milk at higher than normal temperatures and/or salt concentrations as the expression product of a sequence covered by SEQ ID No: 1 and SEQ D No: 2.

The protein may also have deletions, insertions or substitutions of amino acid residues which produce a silent change and result in a functionally equivalent polypeptide. Deliberate amino acid substitutions may be made on the basis of similarity in polarity, charge, solubility, hydrophobicity, hydrophilicity, and/or the amphipathic nature of the residues as long as the biological activity of the polypeptide of the present invention is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below.

Amino acids in the same block in the second column and preferably in the same line in the third column may be substituted for each other:

| | | |
|---|---|---|
| ALIPHATIC | Non-polar | G A P |
| | | I L Y |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-aminobutyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L- Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues.

Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-inks, formation of cystine, formation of pyroglutamate, formylation, gamma-carboxylation, hydroxylation, methylation, oxidation, proteolytic processing, selenoylation, sulfation.

The polypeptide that can be used in the method of the present invention may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals (Porath J (1992) Protein Expr Purif 3-26328 1), protein A domains that allow purification on immobilized immunoglobulin, and the domain utilized in the FLAGS extension/affinity purification system (Immunex Corp, Seattle, Wash.). The inclusion of a cleavable linker sequence such as Factor XA or enterokinase (Invitrogen, San Diego, Calif.) between the purification domain and the polypeptide that can be used in the method of the present invention is useful to facilitate purification.

Nucleotide Sequence.

The terms “variant”, “homologue”, “derivative” or “fragment” in relation to the nucleotide sequence that can be used in the method of the present invention include any substitution of, variation of, modification of, replacement of, deletion of or addition of one (or more) nucleic acid from or to the sequence providing the expression product of the resultant nucleotide sequence the capacity to allow lactic acid bacteria to ferment milk at higher temperatures and/or salt concentrations than normal, preferably having at least the same capacity to allow lactic acid bacteria to ferment milk at higher temperatures and/or salt concentrations as the expression product of a sequence covered by SEQ ID No: 3 and/or SEQ D No: 4.

In particular, the term “homologue” covers identity with respect to structure and/or function providing the expression product of the resultant nucleotide sequence has the capacity to allow lactic acid bacteria to ferment milk at temperatures and salt concentrations higher than normal. With respect to sequence identity (i.e. similarity), preferably there is at least 75%, more preferably at least 85%, more preferably at least 90% sequence identity. More preferably there is at least 95%, more preferably at least 98%, sequence identity. These terms also encompass allelic variations of the sequences.

Sequence identity with respect to SEQ ID No: 3 and/or SEQ ID No: 4 can be determined by a simple “eyeball” comparison (i.e. a strict comparison) of any one or more of the sequences with another sequence to see if that other sequence has, for example, at least 75% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. For a discussion of basic issues in similarity searching of sequence databases, see Altschul et al., 1994, Nature Genetics 6:119-129.

The five BLAST programs available at http://www.ncbi.nlm.nih.gov perform the following tasks:
blastp—compares an amino acid query sequence against a protein sequence database.
blastn—compares a nucleotide query sequence against a nucleotide sequence database.
blastx—compares the six-frame conceptual translation products of a nucleotide query sequence (both strands) against a protein sequence database.
tblastn—compares a protein query sequence against a nucleotide sequence database dynamically translated in all six reading frames (both strands).
tblastx—compares the six-frame translations of a nucleotide query sequence against the six-frame translations of a nucleotide sequence database.

BLAST uses the following search parameters:
HISTOGRAM—Display a histogram of scores for each search; default is yes. (See parameter H in the BLAST Manual).
DESCRIPTIONS—Restricts the number of short descriptions of matching sequences reported to the number specified; default limit is 100 descriptions. (See parameter V in the manual page).
EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).
CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.
ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).
MATRIX—Specify an alternate scoring matrix for BLASTP, BLASTX, TBLASTN and TBLASTX. The default matrix is BLOSUM62 (Henikoff & Henikoff, 1992 Proc Natl Acad Sci USA. 89(22), 10915-9). The valid alternative choices include: PAM40, PAM120, PAM250 and IDENTITY. No alternate scoring matrices are available for BLASTN; specifying the MATRIX directive in BLASTN requests returns an error response.
STRAND—Restrict a TBLASTN search to just the top or bottom strand of the database sequences; or restrict a BLASTN, BLASTX or TBLASTX search to just reading frames on the top or bottom strand of the query sequence.
FILTER—Mask off segments of the query sequence that have low compositional complexity, as determined by the SEG program of Wootton & Federhen 1993 Computers and Chemistry 17:149-163, or segments consisting of short-periodicity internal repeats, as determined by the XNU program of Claverie & States 1993 Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Should Gap Penalties be used when determining sequence identity, then preferably the following parameters are used:

| FOR BLAST | |
|---|---|
| GAP OPEN | 0 |
| GAP EXTENSION | 0 |

| FOR CLUSTAL | DNA | PROTEIN | |
|---|---|---|---|
| WORD SIZE | 2 | 1 | K triple |
| GAP PENALTY | 10 | 10 | |
| GAP EXTENSION | 0.1 | 0.1 | |

Other computer program methods to determine identify and similarity between the two sequences include but are not limited to the GCG program package (Devereux et al., 1984 Nucleic Acids Research 12: 387) and FASTA (Atschul et al., 1990 J Molec Biol 215: 403410).

The present invention also encompasses sequences that are complementary to the sequences that can be used in the method of the present invention or sequences that are capable of hybridising either to the sequences of the present invention SEQ ID No: 3 and SEQ ID No: 4 or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences than can be used in the method of the present invention (including complementary sequences of those presented herein).

The present invention also relates to nucleotide sequences that are complementary to sequences that can hybridise to the nucleotide sequences that can be used in the method of the present invention (including complementary sequences of those presented herein).

Also included within the scope of the present invention are polynucleotide sequences that are capable of hybridising to the nucleotide sequences presented herein under conditions of intermediate to maximal stringency.

The term “selectively hybridizable” means that the nucleotide sequence, when used as a probe, is used under conditions where a target nucleotide sequence that can be used in the method of the invention is found to hybridize to the probe at a level significantly above background. The background hybridization may occur because of other nucleotide sequences present, for example, in the cDNA or bacterial genome DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence that can be used in the method of the present invention under stringent conditions (e.g. 65° C. and 0.1×SSC {1×SSC=0.15 M NaCl, 0.015 M Na₃ Citrate pH 7.0). Where the nucleotide sequence that can be used in the method of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences that can be used in the method of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing CDNA libraries made from or genomic DNA libraries from other animal species, and probing such libraries with probes comprising all or part of the nucleotide sequence set out in SEQ ID No: 3 or SEQ ID No: 4 of the sequence listings of the present invention under conditions of medium to high stringency.

Variants and strain/species homologues may also be obtained using degenerate PCR which will use primers designed to target sequences within the variants and homologues encoding conserved amino acid sequences within the sequences that can be used in the method of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID No: 3 or SEQ ID No: 4 of the sequence listings of the present invention. This may be useful where for example silent codon changes are required to sequences to optimise codon preferences for a particular host cell in which the nucleotide sequences are being expressed.

The nucleotide sequences that can be used in the method of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence that can be used in the method of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from a bacterial cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the polypeptide of the present invention. As will be understood by those of skill in the art, for certain expression systems, it may be advantageous to produce the polypeptides that can be used in the method of the present invention encoding nucleotide sequences possessing non-naturally occurring codons. Codons preferred by a particular prokaryotic cell (Murray E et al., 1989, Nuc Acids Res 17: 477-508) can be selected, for example, to increase the rate of the channel expression or to produce recombinant RNA transcripts having desirable properties, such as a longer half-life, than transcripts produced from naturally occurring sequence.

In one embodiment, the polypeptide that can be used in the method of the present invention, is a recombinant polypeptide.

Preferably the recombinant polypeptide that can be used in the method of the present invention is prepared using a genetic vector.

Constructs

The term “construct” includes the nucleotide sequences described herein, directly attached to a promoter. The same is true for the term “fused” in relation to the present invention which includes direct attachment In each case, the terms do not cover the natural combination of the nucleotide sequence coding for the protein ordinarily associated with the wild type gene promoter and when they are both in their natural environment

The construct may even contain or express a marker which allows for the selection of the genetic construct in, for example, a bacterium, preferably of the genus Escherichia, such as Escherichia coli, or Streptococcus, such as Streprococcus thermophilus, or Lactococcus such as Lactococcus lactis, Lactococcus lactis subsp. cremoris, Lactococcus lactis subsp. Lactis, Leuconostoc, Pediococcus or Bifidobacterium. Various markers exist which may be used, such as markers that provide for antibiotic resistance—e.g. resistance to, kanamycin, gentamycin and ampicillin, penicillin

Preferably the construct of the present invention comprises at least the nucleotide sequence that can be used in the method of the present invention operably linked to a promoter.

Vectors

The term “vector” includes expression vectors, transformation vectors and episomes.

The term “expression vector” means a construct capable of in vivo or in vitro expression.

Preferably, the vectors of the present invention are plasmids.

The vectors of the present invention may be transformed into a suitable host cell as described below under controlled conditions to provide a suitable environment for expression of a polypeptide that can be used in the method of the present invention. Thus, in a further aspect the invention provides a process for expressing polypeptides according to the present invention which comprises cultivating a host cell transformed with an expression vector as described above under conditions to provide for expression by the vector of a coding sequence encoding the polypeptides that can be used in the method of the present invention.

Control sequences operably linked to sequences encoding the protein that can be used in the method of the invention include promoters/enhancers and other expression regulatory sequences. These control sequences may be selected to be compatible with the host cell for which the expression vector is designed to be used for. The term promoter is well-known in the art e.g. as an RNA polymerase binding site and encompasses nucleic acid regions ranging in size and complexity from minimum promoters to promoters which include upstream regulatory sequences and enhancers.

The term “promoter” is used in the normal sense of the art, e.g. an RNA polymerase binding site. The promoter may optionally contain an enhancer element.

The term “enhancer” includes a DNA sequence which binds to other protein components of the transcription initiation complex and thus facilitates the initiation of transcription directed by its associated promoter.

The promoter can include features to ensure or to increase the level of expression in a suitable host. For example, the features can be conserved regions such as a Pribnow Box, Kozak sequence or a TATA box. The promoter may even contain other sequences to affect (such as to maintain, enhance, decrease) the levels of expression of the nucleotide sequence. For example, suitable other sequences include: target sequences for transcription regulating proteins, expended—10 motifs, RNA leader sequences, UP elements and others. Other sequences include inducible elements—such as temperature, chemical, light or stress inducible elements. Also, suitable elements to enhance transcription or translation may be present.

In another embodiment, a constitutive promoter may be selected to direct the expression of the desired polypeptide that can be used in the method of the present invention. Such an expression construct may provide additional advantages since it circumvents the need to culture the expression hosts on a medium containing an inducing substrate.

The promoter is typically selected from promoters which are functional in bacterial or fungal cells referred to as prokaryotic promoters and eukaryotic promoters.

More preferably, the promoter is selected from promoters which are active in lactic acid bacteria.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

The vectors of the present invention may contain one or more selectable marker genes for example an ampicillin or penicillin resistance genes.

The preferred selection systems for industrial micro-organisms are those formed by the group of selection markers which do not require a mutation in the host organism. Examples of fungal selection markers are the genes for acetamidase (amdS), ATP synthetase, subunit 9 (oliC), orotidine-5′-phosphate-decarboxylase (pvrA), phleomycin and benomyl resistance (benA). Examples of non-fungal selection markers are the bacterial G418 resistance gene (this may also be used in yeast, but not in filamentous fungi), the ampicillin or kanamycin resistance genes (E. coli), the neomycin resistance gene (Bacillus) and the E. coli uidA gene, coding for β-glucuronidase (GUS).

Vectors may be used in vitro, for example for the production of RNA or used to transfect, a host cell.

The vectors of the present invention also comprises episomes.

As used herein the term ‘episome’ means a unit of genetic material composed of a series of genes which optionally comprises a promoter that sometimes has an independent existence, as an extrachromosomal unit, in a host cell and at other times is integrated into a chromosomes of the cell, replicating itself along with the chromosome. Episomes have been studied in bacteria. One group of episomes are actually viruses that infect bacteria Episomes called sex factors determine whether chromosome material will be transferred from one bacterium to another. Other episomes carry genes that make bacteria resistant to the inhibitory action of antibiotics. Furthermore, episomes carry genes that enable bacteria to survive higher than regular temperatures, acidity, salt etc.

Thus, polynucleotides that can be used in the method of the present invention can be incorporated into a recombinant vector (typically a replicable vector), for example a cloning or expression vector. The vector may be used to replicate the nucleic acid in a compatible host cell. Thus in a further embodiment, the invention provides a method of making polynucleotides that can be used in the method of the present invention by introducing a polynucleotide into a replicable vector, introducing the vector into a compatible host cell, and growing the host cell under conditions which bring about replication of the vector. The vector may be recovered from the host cell. Suitable host cells are described below in connection with expression vectors.

Cells

The present invention includes within its scope cells comprising nucleic acid molecules, constructs or vectors of the present invention. These may for example be used for expression, as described herein.

Host cells transformed with the nucleotide sequence encoding the polypeptide that can be used in the method of the present invention may be cultured under conditions suitable for the expression and recovery of the polypeptide from cell culture. The protein produced by a recombinant cell may be secreted or may be contained intracellularly depending on the sequence and/or the vector used. As will be understood by those of skill in the art, expression vectors containing nucleotide sequences coding for the polypeptide that can eused in the method of the present invention can be designed with signal sequences which direct secretion of the polypeptide through a particular prokaryotic cell membrane. Other recombinant constructions may join the nucleotide sequence coding for the polypeptide that can be used in the method of the present invention to nucleotide sequence encoding a polypeptide domain which will facilitate purification of soluble proteins (Kroll D J et al., 1993, DNA Cell Biol 12:441-53, see also above discussion of vectors containing fusion proteins).

Cells that can be used in the method of the present invention may be provided in any appropriate form. For example, they may be provided in isolated form, in culture, in stored form, etc. Storage may, for example, involve cryopreservation, buffering, sterile conditions, etc. Cells that can be used in the method of the present invention may be provided by gene cloning techniques, or by any other means.

Preferably the nucleotide sequence that can be used in the method of the present invention is operably linked to a transcription unit.

The term “transcription unit(s)” as described herein are regions of nucleic acid containing coding sequences and the signals for achieving expression of those-coding sequences independently of any other coding sequences. Thus, each transcription unit generally comprises at least a promoter, an optional enhancer and a polyadenylation signal.

Hybrid promoters may also be used to improve inducible regulation of the expression construct.

The term “transformed cell” means a cell having a modified genetic structure. With the present invention, a cell has a modified genetic structure when a vector according to the present invention has been introduced into the cell.

The present invention also provides a method comprising transforming a host cell with the nucleotide sequence that can be used in the method of the present invention.

The present invention also provides a method comprising culturing a transformed host cell—which cell has been transformed with a nucleotide sequence according to the present invention under conditions suitable for the expression of the polypeptides that can be used in the method of the present invention encoded by said nucleotide sequences as described herein.

The present invention also provides a method comprising culturing a transformed host cell—which cell has been transformed with a nucleotide sequence according to the present invention or a derivative, homologue, variant or fragment thereof—under conditions suitable for the expression of the polypeptide that can be sued in the method of the present invention encoded by said nucleotide sequence; and then recovering said polypeptide from the transformed host cell culture.

The present invention also encompasses nucleotide sequences that are complementary to the sequences presented herein, or any fragment or derivative thereof If the sequence is complementary to a fragment thereof then that sequence can be used as a probe to identify similar promoter sequences in other organisms.

The polypeptides that can be used in the method of the present invention and fragments thereof may be produced in recombinant E. coli or fungi. Preferably, polypeptides for use in the method of the present invention and fragments thereof are produced in recombinant lactic acid bacteria

The term “transformation vector” means a construct capable of being transferred from one entity to another entity—which may be of the species or may be of a different species. If the construct is capable of being transferred from one species to another—such as from an E.coli plasmid to a bacterium, such as of the genus Streptococcus, Lactococcus, Lactobacillus, Leuconostoc, Bifidobacterium or Pediococcus then the transformation vector is sometimes called a “shuttle vector”.

The term “host cell”—in relation to the present invention includes any cell that could comprise the nucleotide sequence coding for the recombinant protein according to the present invention and/or products obtained therefrom, wherein a promoter can allow expression of the nucleotide sequence according to the present invention when present in the host cell. Furthermore, vectors and polynucleotides of the present invention (the reporter gene constructs described above) may also be introduced into host cells for replicating the vector/polynucleotide.

In a prefererd embodiment, the host cell is a lactic acid bacterium. By way of example the lactic acid bacteria can be selected from a group of bacteria which consists but is not limited to Streptococcus, Lactococcus, Lactobacillus, Leuconostoc, Bifidobaterium or Pediococcus.

Transformation of Host Cell/Host Organisms

As indicated earlier, the host organism can be a prokaryotic or an eukaryotic organism. Examples of suitable prokaryotic hosts include E. coli. More preferably lactic acid bacteria such as Streptococcus, Lactococcus, Lactobacilus, Pediococcus, Leuconostoc or Bifidobacterium. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

Although the presence/absence of marker gene expression suggests that the nucleotide sequence and/or the polypeptide that can be used in the method of the invention is also present, the presence and expression should be confirmed. For example, if the nucleotide sequence coding for the polypeptide that can be used in the method of the present invention is inserted within a marker gene sequence, recombinant cells containing the polypeptide coding regions may be identified by the absence of marker gene function. Alternatively, a marker gene may be placed in tandem with the nucleotide sequence coding for the polypeptide that can be used in the method of the present invention under the control of a single promoter. Expression of the marker gene in response to induction or selection usually indicates expression of the polypeptide as well.

Additional methods to quantitate the expression of a particular molecule include radiolabelling (Melby P C et al., 1993, J Immunol Methods 159: 23544) or biotinylating (Duplaa C et al., 1993, Anal Biochem 229-36) nucleotides, coamplification of a control nucleic acid, and standard curves onto which the experimental results are interpolated. Quantitation of multiple samples may be speeded up by running the assay in an ELISA format where the polypeptide that can be used in the method of the present invention is prepared in various dilutions and a spectrophotometric or calorimetric response gives rapid quantitation.

The present invention also relates to expression vectors and transformed host cells comprising nucleotide sequences coding for the polypeptide that ca be used in the method of the invention or variant, homologue, fragment or derivative thereof for the in vivo or in vitro production of the polypeptide with a view to ferment milk which leads to the profuction of different milk products according to the present invention.

### EXAMPLES

The present invention will now be illustrated by way of examples where reference is made to the following figures and tables.

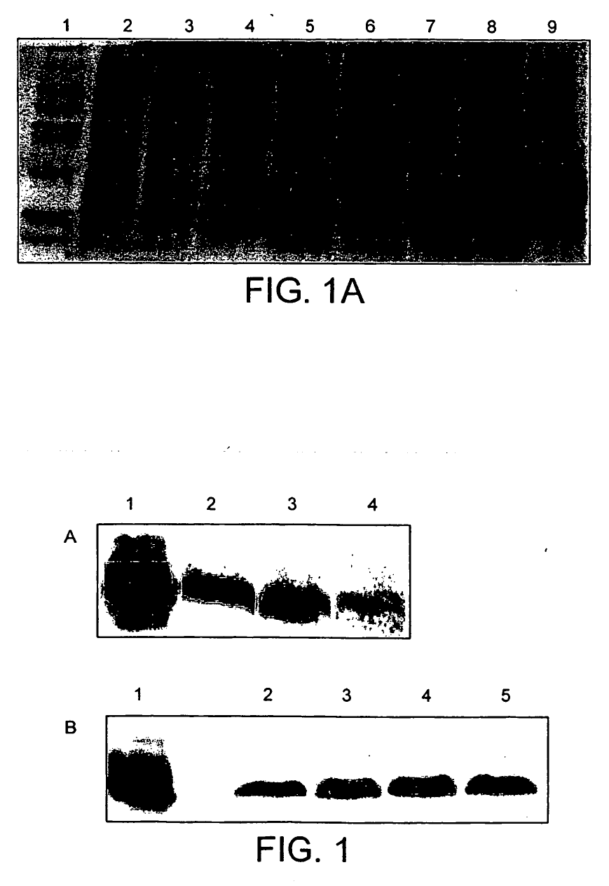

FIG. 1.A. Shows an SDS-PAGE

FIG. 1.B. Shows a Western Blot

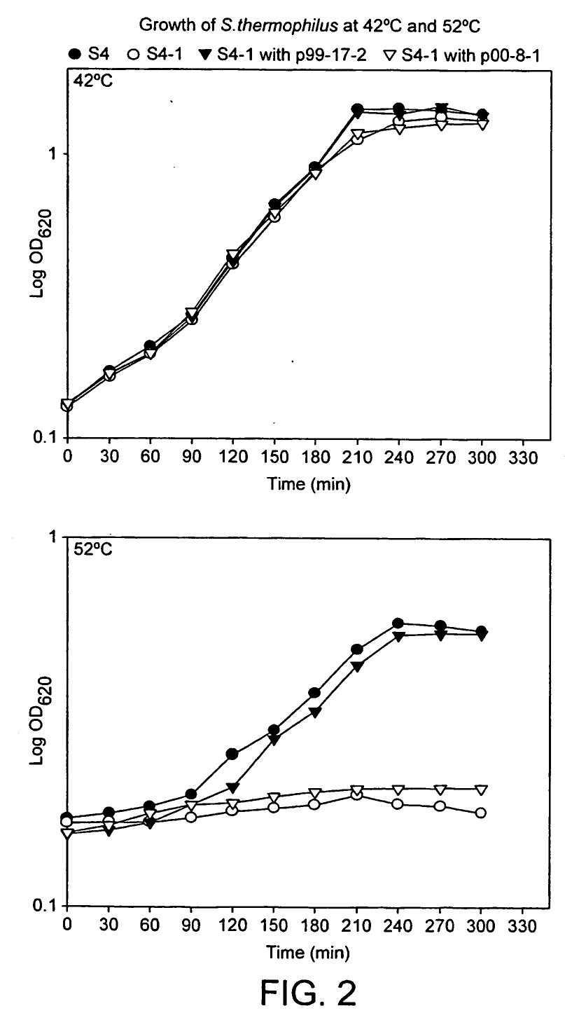

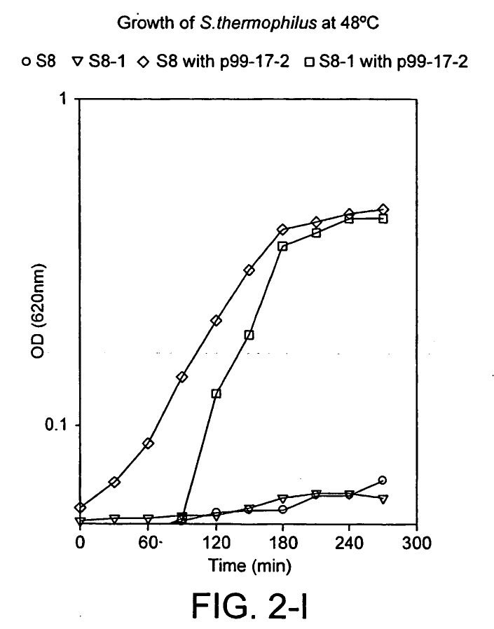

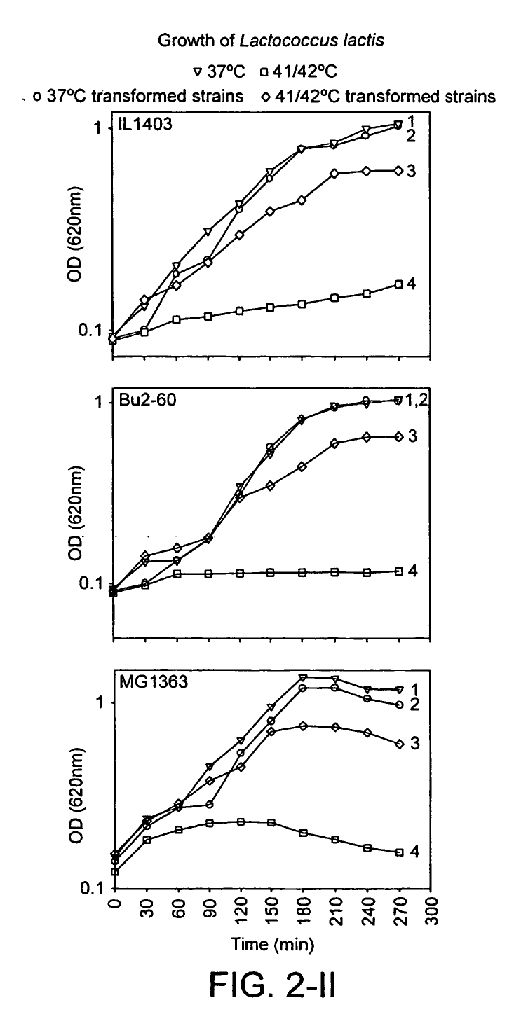

FIG. 2.A. and B. Show a graphs.

FIG. 2-1. Shows a graphic representation of log OD₆₂₀

FIG. 2-2. Shows a graph.

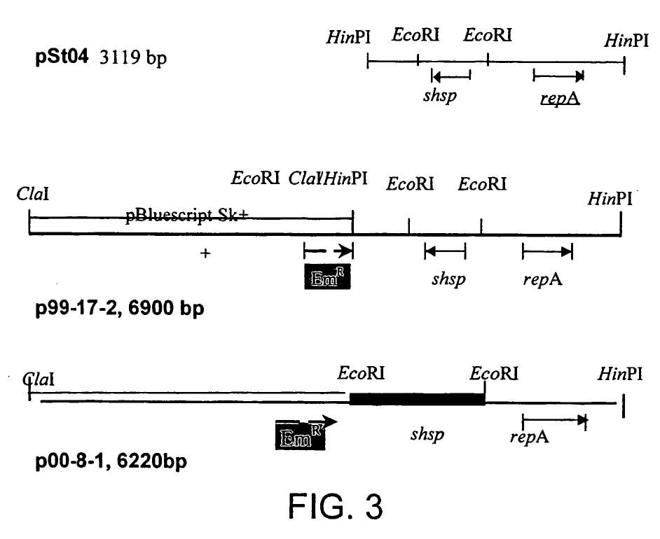

FIG. 3. Shows a schematic.

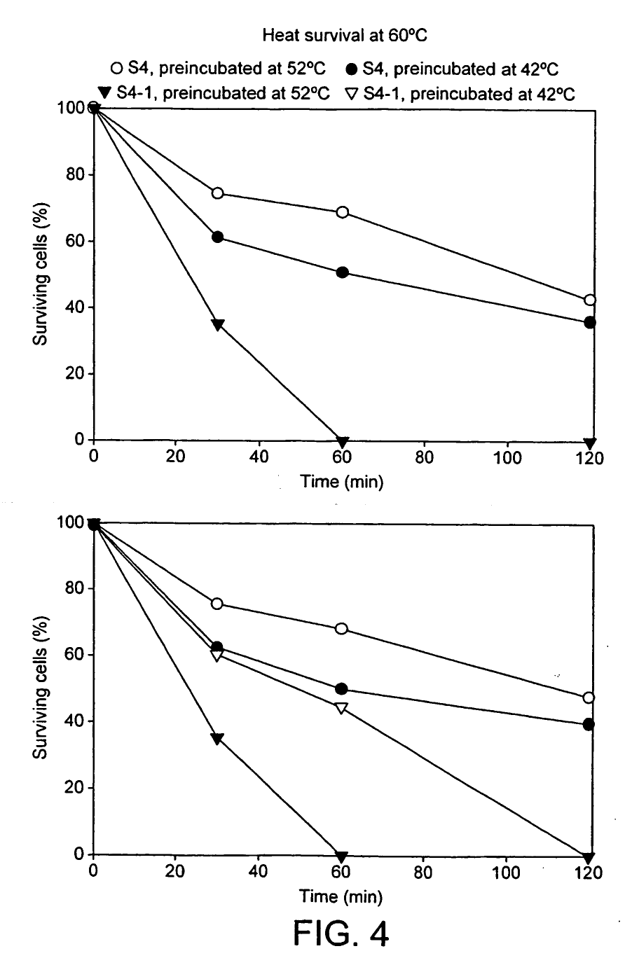

FIG. 4. Shows a graphic representation of % surviving cells

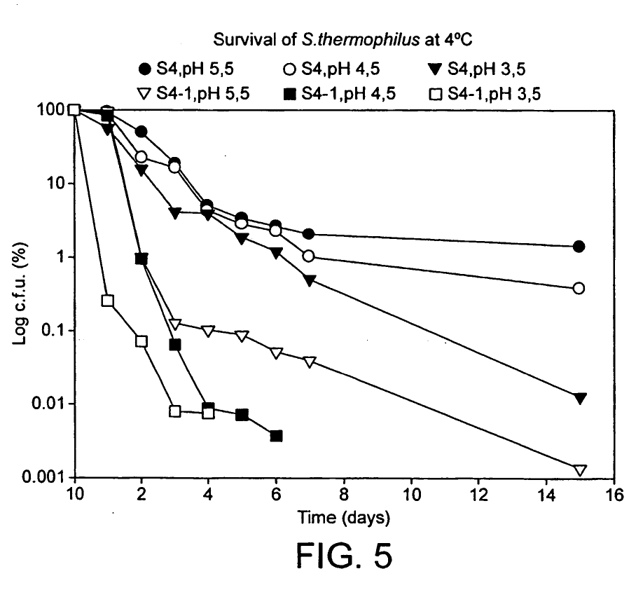

FIG. 5. Shows a graphic representation of log colony forming units (%)

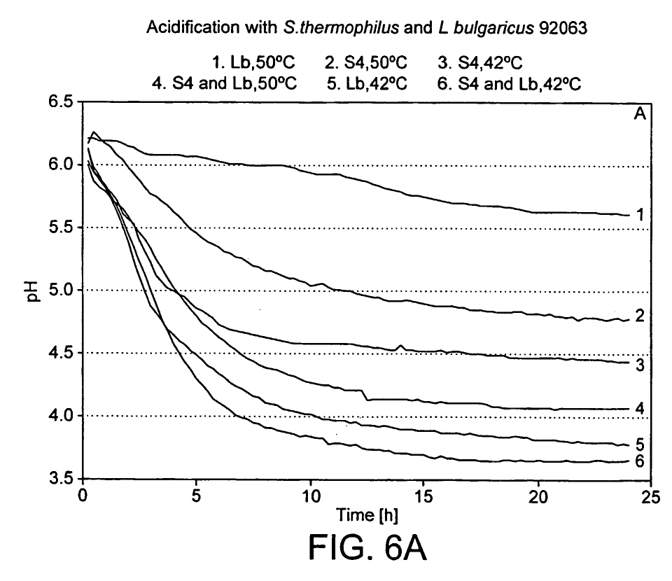

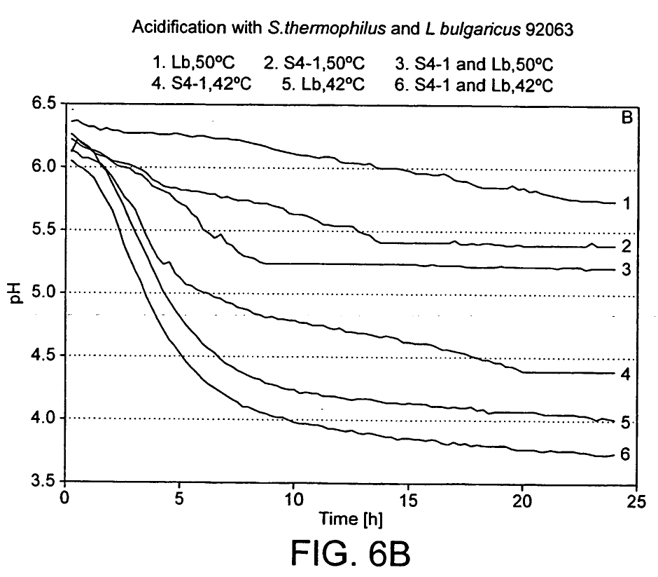

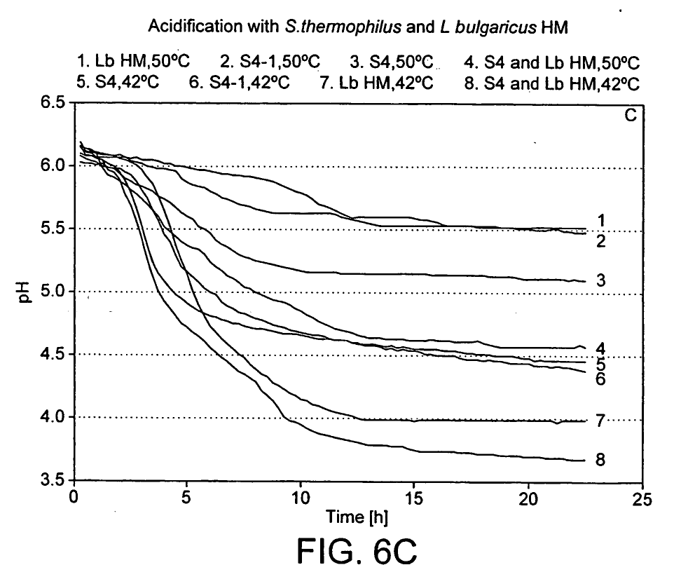

FIG. 6.A. Shows a graph

FIG. 6.B. Shows a graph

FIG. 6.C. Shows a graph

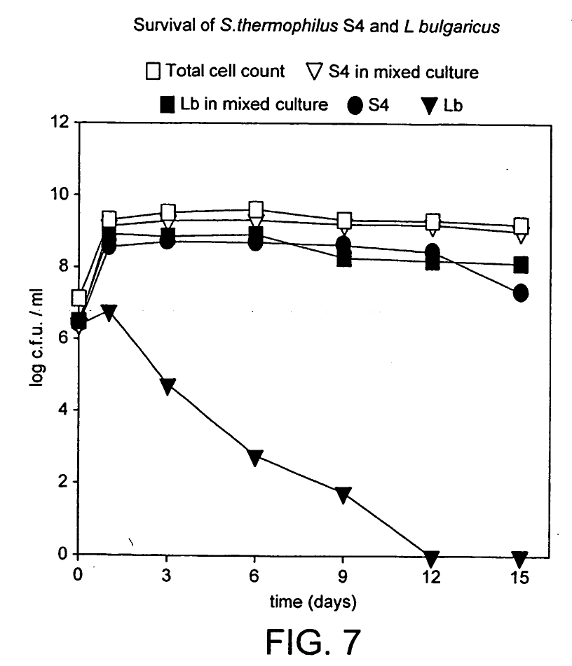

FIG. 7. Shows a graphic representation of log colony forming units/ml

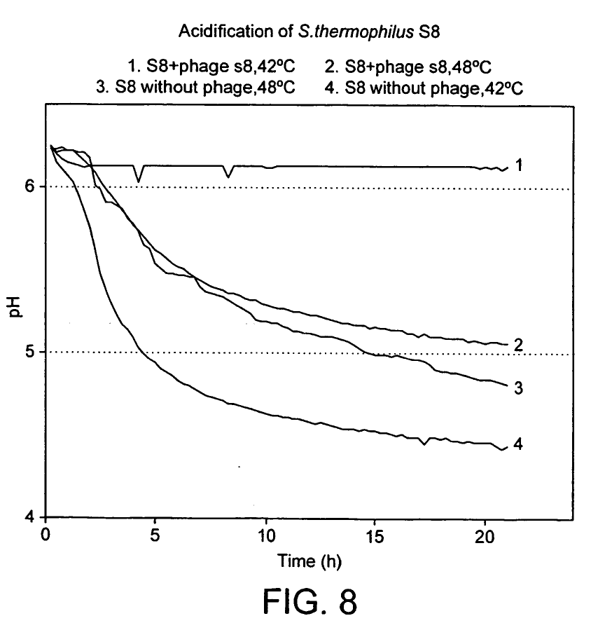

FIG. 8. Shows a graph

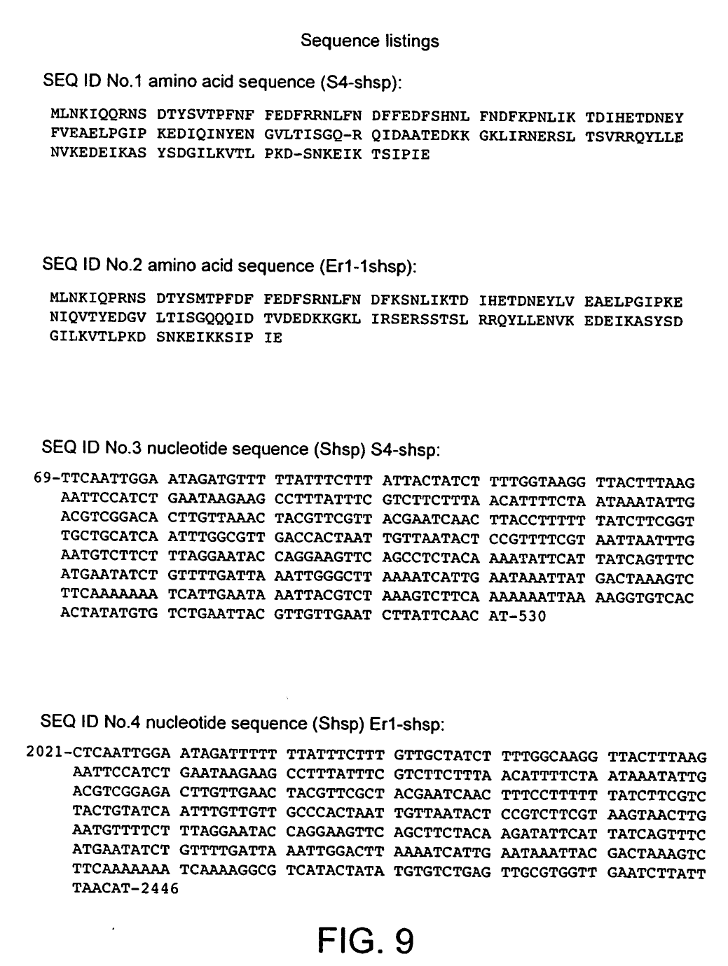

FIG. 9. Shows sequence listings

Table 1. Bacterial strains and bacteriophage

Table 2. Change of the pH-values of yoghurt produced with S. thermophilus S4 and Lactobacilus bulgaricus 92063 at 42° C. and 50° C. during storage at 4° C.

The Figures Explained in Greater Detail.

FIG. 1.A.

SDS-PAGE of the soluble proteins of S. thermophilus S4-1 (lane 2) and S4 w/o and with heat shock after incubation for 60 min at 40° C. (lanes 3 and 4), 45° C. (lanes 5 and 6) and 55° C. (lanes 7 and 8). Lane 1 contains protein size markers with molecular masses of 6.5; 14.2; 18.4; 29; 48.5; and 66 kDa. Lane 9 contains partially purified sHsp isolated from an overexpressing E. coli strain. The heat shock was performed at 52° C. for 30 min prior to incubation at the indicated times.

FIG. 1.B. Induction of sHsp expression in S. thermophilus S4. Cells of S. thermophilus S4 and S4-1 grown in LTM17 medium to the late log-phase of growth (OD_{620˜1.0}) were collected, washed with sugar-free medium, and resuspended in TM17 containing 0.05% glucose. 5ml aliquots were incubated for 30 min at 37, 46, and 52° C. (A) or at 52° C. for 5-60 min (B). Cell extracts were prepared and Western blot analysis was performed. A: partially purified sHsp from E. coli (positive control), lane 1; S. thermophilus S4 incubated at 52, 46, and 37° C., lanes 2 to 4; S4-1 incubated at 52° C. (negative control), lane 5. B: positive control, lane 1; 5 min, lane 2; 15 min, lane 3; 30 min, lane 4; 60 min, lane 5.

FIG. 2.A. Growth curves showing S. thermophilus S4 (filled circles, (● S4)), its plasmid-cured derivative S4-1 (open circles, (∘ S4-1)), S4-1 transformed with the recombinant plasmids p99-17-2 (shsp⁺) (filled triangles, (▾ S4-1)), and p00-8-1 (shsp⁻)(open triangles, (∇ S4-1)) incubated at 42° C.

FIG. 2.B. Growth curves showing S. thermophilus S4 (filled circles, (● S4)), its plasmid-cured derivative S4-1 (open circles, (∘ S4-1)), S4-1 transformed with the recombinant plasmids p99-17-2 (shsp⁺)(filled triangles, (▾ S4-1)), and p00-8-1 (shsp⁻)(open triangles, (∇ S4-1)) incubated at 52° C.

FIG. 2-1: Growth at 48° C. of Streptococcus thermophilus S8 (circles, (∘ S8)), its plasmid-free derivative S8-1 (triangles, (∇ S8-1)) and of strains S8 and S8-1 transformed with the recombinant plasmid p99-17-2 (diamonds, squares, (⋄ S8 with p99-17-2, □ S8-1 with p99-17-2).

FIG. 2-2: Growth of Lactococcus lactis strains Bu2-60, IL1403, and MG1363 and its derivatives transformed with the recombinant (shsp+) plasmid p99-17-2. Bu2-60 IL1403, MG1363 at 37° or 41/42° C. (1-triangles and 4-squares, (∇ 37° C. and □ 41/42° C.)); transformed strains at 37 or 41/42° C. (2-circles and 3-diamonds, (∘ 37° C. and ⋄ 41/42° C.)).

It is seen from the figures that all the Lactococcus lactis strains grow well at 37° C. (1) but not at 42° C. (4). The strains transformed with the plasmid p00-17-2 increases growth at 42° C. (3) whereas growth at 37° C. does not change.

FIG. 3. Physical maps of the plasmids pSt04, p99-17-2, and p00-8-1.

FIG. 4. Heat survival curves of S. thermophilus S4 (circles, (∘) S4, preincubated at 52° C. and (●) S4, preincubated at 42° C.)) and its plasmid-free derivative S4-1 (triangles, (∇) S4-1, preincubated at 52° C. and (▾) S4-1, preincubated at 42° C.) in TM17 medium at 60° C. Prior to exposure to 60° C., cells were preincubated at 42° C. (filled symbols, (●) S4 and (▾) S4-1)) or 52° C. (open symbols, (∘) S4 and (∇) S4-1)) for 30 min.

It is seen from FIG. 4 that S4 shows better survival than the plasmid free strains S4-1.

FIG. 5. Survival of S. thermophilus S4 and its plasmid-free derivative S-1 during storage at 4° C. at different pH-values. Cells were resuspended in growth medium preadjusted to the appropriate pH by addition of lactic acid and stored at 4° C. up to 15 days. The cell count was determined every third day by plating proper dilutions on LTM17 agar. (●) S4, pH 5,5; (∘) S4, pH 4,5; (▾) S4, pH 3,5; (▾) S4-1, pH 5,5; (▪) S4-1, pH 4,5; (□) S4-1, pH 3,5.

From the Figure it can be seen that S4 exhibits better survival at all the mentioned pH values. The survival of S4-1 is dramatically reduced after 1-2 days storage and after 4-6 days almost no bacteria have survived.

FIG. 6. Skim milk acidification in single and mixed strain fermentations with S. thermophilus S4 and S4-1 and L. bulgaricus 92063 at different temperatures. Skim milk was inocculated with 5×10⁶ CFU/ml for each strain in the mixed strain and 10⁷ CFU/ml for the single strain fermentations. The decrease of the pH was followed automatically with a multichannel pH-meter.

FIG. 6.A: Lb 92063 at 50° C. (1); S4 at 50° C. (2); S4 at 42° C. (3); S4 and Lb at 50° C. (4); Lb at 42° C. (5); S4 and Lb at 42° C. (6). following:

FIG. 6.B: Lb 92063 at 50 C. (1); S4-1 at 50° C. (2); S4-1 and Lb at 50° C. (3); S4-1 at 42° C. (4); Lb at 42° C. (5); S4-1 and Lb at 42° C. (6).

FIG. 6.C: Lb HM at 50° C. (1); S4-1 at 50° C. (2); S4 at 50° C. (3); S4 and Lb HM at 50° C. (4); S4 at 42° C. (5); S4-1 at 42° C. (6); Lb HM at 42° C. (7); S4 and Lb HM at 42° C. (8).

From FIG. 6A and B it is seen that curve no 6 in both figures corresponds to a normal yoghourt fermentation. After 15-20 hours fermentation a pH of around 3.6 is reached. Curve 4 in FIG. 6A and 6C shows the fermentation carried out at 50 C. where the Streptococcus thermophilus carries the shsp. These fermentations reach a pH level around 4.0-4.1. Curve 3 in FIG. 6 B shows that fermentation with S4-1 and Lb 92063 ends at a pH around 5.2.

FIG. 7. Survival of S. thermophilus S4, and L. bulgaricus after growth in single or mixed strain culture at 50° C. and subsequent storage. Skim milk was inoculated to final cell density of 10⁷ CFU/ml and incubated for 24 H at 50° C. Subsequently the fermented milk was stored at 4° C. for 15 days. The total cell count for each strain as well as the total cell count (counts of Lactobacillus and S4 together, was determined every third day. Total count (open squares, (□)); S4 and Lb in mixed culture (open triangles (∇ S4) and filled squares (▪ Lb)); S4 (filled circles, (●) S4); Lb (filled triangles, (▾) Lb).

FIG. 8. Acidification of S. thermophilus S8 and its transformed derivative S8 (pSt04-shsp⁺) at 42° and 48° C. Skim milk was inoculated with 10⁷ CFU/ml and incubated at the indicated temperature in the absence or presence of bacteriophage s8 at a multiplicity of infection (m.o.i.) of 10. Acidification was followed automatically with a multichannel pH-meter. S8 (pSt04)+phage s8, 42° C. (1); S8 (pSt04)+phage s8, 48° C. (2); S8(pSt04) w/o phage, 48° C. (3); S8 (pSt04) w/o phage, 42° C. (4).

FIG. 9. Sequence listings.

SEQ ID No: 1

SEQ ID No: 2

SEQ ID No: 3

SEQ ID No: 4

Specific Examples.

General Features of Plasmids pSt04 and pER1-1 Carrying Genes for Small Heat Shock Proteins.

The nucleotide sequence of plasmids pSt04 and pER1-1, which belong to DNA-homology group I of plasmids from Streptococcus thermophilus (Janzen et al. 1992) were determined (Accession numbers: AJ 242477 and AJ 242476). These plasmids share a DNA region of about 1200 bp with homologies of more than 90% which is necessary for plasmid replication.

In addition to the repA gene, plasmids pSt04 and pER1-1 each possess a second ORF encoding a polypeptide of 155 and 142 amino acids (aa) residues (SEQ ID No: 1 and SEQ ID No: 2) respectively, corresponding to molecular masses of 18,042 and 16,422 Da These ORFs are positioned counter clockwise to the repA-genes. Putative ribosomal binding sites (GAa/gGAAAG) 8 bp upstream of the start-codons are preceeded by 5′-TTGAAA . . . (16 bp) . . . TATAAT promoter regions. The predicted gene products are highly similar (>90%) to small heat shock proteins (sHsp) observed in other S. thermophilus strains (O'Sullivan et al., 1999, Somkuti et al., 1998). These sHsps belong to a family of shock response proteins common in prokaryotic and eukaryotic organisms. The sHsp synthesis significantly increases from a low basic level at pH-values below pH 4.5 or at temperatures above 45° C. and could be further increased by a short, 30 min shock at pH 4.0. or at 52° C. (FIG. 1).

Functions of Small Heat Shock Proteins.

i) Growth at Elevated Temperatures and Thermoresistance

The growth behaviour at different temperatures of S. thermophilus strains S4 and ER1, carrying plasmids pSt04 and pER1-1, respectively, was compared to that of the plasmid-cured derivatives S4-1 and ER1-1 of these strains. At 42° C. all strains grew with identical rates to final cell densities of about 2×10⁹ CFU/ml. At 45° C. the plasmid-free strains grew somewhat slower but to almost the same final cell densities. At 52° C. only the plasmid (shsp)-bearing strains were able to grow with reduced growth rates to final cell densities of about 1×10⁹ (FIG. 2).

To verify that the shsp-gene was responsible for the ability to grow at high temperature, plasmid-free strain S4-1 was transformed with recombinant plasmids p99-17-2 and p00-8-1. Plasmid p99-17-2 contains the entire pSt04 sequence cloned into the E. coli vector pBluescriptII (SK+) and carries as selection marker for gram-positive bacteria the erythromycin-resistance gene from the Staphylococcus aureus plasmid pE194. Plasmid p00-8-1 is a deletion derivative of p99-17-2 missing the entire shsp-sequence (FIG. 3). Strains S4-l(p99-17-2) and S4-1(p00-8-1) showed the same growth behaviour as S4 and S4-1, respectively, proving that the presence of the shsp-gene was responsible for the ability to grow at temperatures up to 52° C. (FIG. 2).

The recombinant plasmid p99-17-2 was subsequently transfered by electroporation into S. thermophilus strains S8 and S11 as well as into the mesophilic Lactococcus lactis subsp. lactis strains IL1403 and Bu2-60 and L. lactis subsp.cremoris strain MG1363. The maximal growth temperatures for S8 and S11 were raised from 45° C. up to 48° C. (FIG. 2-1) and 52° C. (FIG. 2), respectively and from 37° C. up to 42° C. for the lactococcal strains (FIG. 2-2).

Compared to its plasmid-free derivatives S. thermophilus strains 54 and ER1 exhibited higher thermoresistance. About 50% of the plasmid-bearing but non of the plasmid-free cells survived incubation at 60° C. for 2 h. By a short heat-shock (30 min at 52° C.) the thermoresistance could be further improved (FIG. 4).

ii) Acid Tolerance

In addition to an increased maximal growth temperature and improved thermoresistance the presence of sHsp increased the acid tolerance. Cells expressing shsp showed a strongly improved viability upon storage at low pH. Even at pH 3.5, a pH value lower than in most fermented milk products, more than 40% of the cells survived storage for 15 days at 4° C., whereas no cells of the shsp-negative mutants were detectable after 7 days at identical conditions (FIG. 5).

iii) Salt Tolerance.

S. thermophilus S4 and its plasmid-free derivative S4-1 were grown at sodium chloride concentrations from 0-3%. No obvious differences either in growth rate or the final cell densities were observed at NaCl-concentrations up to 1%. At salt concentrations from 1.5 to 2.5% the wildtype strain grew faster than the plasmid-cured strain. No growth occured at salt concentrations of 3% NaCl.

Application of Thermoresistant S. thermophilus Strains in Mixed Culture with L. bulgaricus for Milk Fermentation.

S. thermophilus strains carrying a plasmid-encoded shsp-gene show significantly increased thermo- and acid-resistence. To prove, whether the two available shsp-positive S. thermophilus strains, S4 and ER1 , were suitable for yoghurt production, each of the strains was used in combination (1:1) with Lactobacillus delbrueckii subsp. bulgaricus (a. bulgaricus) 92063 and HM for skim milk fermentation. Mixed cultures of L. bulgaricus and isogenic derivatives of S4 and ER1, cured of the shsp-gene containing plasmids, served as a control.

Acidification During Milk Fermentation

The strain combination S4/Lb92063 showed significant protosymbiotic effects in the 42° C. as well as the 50° C. fermentations. At 42° C. the pH dropped to values below pH 4.0 after 7 h and decreased to pH 3.6 after 24 h. At 50° C. the pH-value was 4.3 after 8-10 h and 4.1 after 24 h (FIG. 6A). The strain combination S4-1 with LB 92063 showed significant protosymbiotic effects at 42° C., but no acidification was observed at 50° C. (FIG. 6B).

With the strain combination, S. thermophilus ER1 and Lb 92063, similar results were obtained. The acidification activity was, however, slightly lower. The control fermentations (S. thermophilus ER1-2 and Lb 92063) at 42° C. were very similar to the test-fermentation at this temperature; at 50° C. acidification was unsufficient.

S. thermophilus S4 in combination with L. bulgaricus HM gave similar results as the combination S4/Lb 92063 (FIG. 6C).

Cell Counts During Milk Fermentation and Subsequent Storage

Starting with a cell number of about 2×10⁶ cfu/ml for each strain, the cell number increased in fermentations with the mixed culture S4/Lb92063 at 50° C. to 10⁹ (S4) and 6×10⁸ (Lb) cfu/ml. In single-strain fermentations, strain S4 grew to cell numbers of about 5×10⁸ cfu/ml, whereas the plasmid-cured, shsp-negative strain S4-1 as well as the Lb 92063 grew only slowly to final cell densities of about 8×10⁶. After a 24 h fermentation the fermented milk was stored at 4° C. for 15 days. The cell numbers of the single strains as well as the total cell counts were determined every third day. At the end of the storage period only a slight decrease of the cell numbers was observed for S. thermophilus S4 and and Lb 92063. The high survival of Lb 92063 was particularly surprising, since this strain, grown in single strain culture at 50° C. was no longer detectable after storage for 12 days (FIG. 7). The reason for this positive effect on the survival of L. bulgaricus grown at 50° C. in mixed culture together with a thermo resistant S. thermophilus is still unknown.

Production of Yoghurt with a Mixed-Strain Culture of S. thermophilus S4 and L. bulgaricus 92063 at 42 and 50° C.

Pasteurised milk (3.5% fat) was inoculated with a mixed culture of S4 and Lb 92063 and incubated at 42° C. and 50° C. for 12 h. At the end of the fermentation the yoghurt products were tested for texture, pH, cell number, taste and aroma The yoghurt products were subsequently stored at 4° C. for 2 weeks and tested every third day as above. Yoghurt produced at 42° C. had a firm texture with slight whey separation, a pH-value of 3.6 to 3.5 and cell numbers of about 10⁹ cfu/ml for each of the strains. It tasted very acidic and had a sour aroma Yoghurt produced at 50° C. also showed a firm texture without whey separation, the pH was about 4.3 after fermentation and 4.0 at the end of the storage period (Table.2). The cell numbers were comparable to the 42° C.-fermentation and decreased only slightly during storage. The yoghurt tasted mildly, comparable to a well-fermented “Joghurt-Mild,” and had a rich aroma Fermentation at 50° C. with a proper combination of a thermo resistant S. thermophilus and a L. bulgaricus resulted in a yoghurt with the characteristic of a “Joghurt-Mild”, but with the original microflora of a classical yoghurt and high cell density even after storage for at least the shelf live of the product.

Influence of the Fermentation Temperature on the Bacteriophage Sensitivity.

To test the effect of the fermentation temperature on the phage sensitivity, the acidification activity of S. thermophilus S8 transformed with the shps-plasmid pSt04 was determined at 42 and 48° C. in the absence or presence of the virulent phage s8 at a multiplicity of infection (M.o.i.) of about 10. At 42° C. no acidification was observed, whereas at 48° C. a final pH of about 5.0 was obtained (FIG. 8). This indicates, that even at this unrealistic high phage load higher fermentation temperatures have a protective effect against phage attack.

Material and Methods

Bacterial Strains, Bacteriophages and Media.

The strains used in this study are listed in Table 1. S. thermophilus and Lactococcus lactis were grown in TM 17 medium (Krusch et al. 1987) supplemented with 1% lactose, L. bulgaricus was grown in MRS medium (Merck, Darmstadt, Germany). E. coli was grown at 37° C. in Luria broth (Sambrook et al. 1989). When appropriate, antibiotics were added as follows: 3 or 5 μg/ml erythromycin for S. thermophilus and Lactococcus lactis, respectively, and 100 μg erythromycin per rnl for E. coli. Blue-white selection was performed as described by Sambrook et al.,1989). Acidification was determined in 9% skim milk medium, pH was monitored by an 8-channel pH-meter (Ingenieurbüro Messelektronik, Chemnitz, Germany). Bacteriophage s8 was propageted as described by Sambrook et al.,1989) and added to the fermentation assays at a multiplicity of infection (m.o.i.) of about 10.

DNA Isolation and Manipulation.

Plasmid DNA was isolated from E. coli with a NucleoSpin kit (Macherey-Nagel, Düren, Germany) and from S. thermophilus and L. lactis by adapting the method accordingly. Enzymes were purchased from New England Biolabs, Beverley, Mass. and used according the manufacturer's instructions.

Transformation.

E. coli was electrotransformed by a procedure recommended by Bio-Rad Laboratories, Richmond, Calif., L. lactis as described by Holo and Nes (1989) and S. thermophilus according to Mohamed (PhD-thesis, Univ. Kiel, Germany, 2002).

Construction of Plasmids p99-17-2 and p00-8-1.

Plasmid pSt04 was linearized by digestion with HinP1 I and cloned into pBluescrips II SK+ (ClaI). The recombinant plasmid p99-16-5 was subsequently genetically marked by insertion of a erthromycin-resistance gene cassette into the BamHI site of pBluescript. A small EcoRI fragment was removed resulting in p00-8-1 which lacks the entire shsp-gene. The recombinant plasmids were propagated in E. coli XL-blue and transfered into S. thermopilus by electroporation.

Plasmid Curing.

S. thermophilus strains were cured of their plasmids by protoplast-induced curing essentially as described by Gasson (1983).

References

Chopin, A. M.; Chopin, M. C., Moille-Batt, A. & Langella, P. (1984). Two plasmid-determined restriction and modification systems in Streptococus lactis. Plasmid 11, 260-263
Gasson, J. M. (1983). Plasmid complement of Streptococcus lactis NCDO 712 and other lactic streptococci after protoplast-induced curing. J. Bacteriol. 154, 1-9
Holo, H. & Nes, I. F. (1989). High-frequency transformation, by electroporation, of Lactococcus lactis subsp. cremoris grown with glycine in osmotically stabilized media. Appl. Environ. Microbiol. 55, 3119-3123.
Janzen, T., Kleinschmidt, J., Neve, H. & Geis, A. (1992). Sequencing and characterization of pST1, a cryptic plasmid from Streptococcus thermophilus. FEMS Microbiol Lett 95,175-180
Krusch, U., Neve, H., Luschei, B. & Teuber, M. (1987). Characterization of virulent bacteriophages of Streptococcus salivarius subsp. thermophilus by host specificity and electron microscopy. Kieler Milchwirtsch Forsch Ber 39, 155-167
Mohamed, H. A. M. I. (2002). New techniques for food biotechnology. PhD-Thesis, University Kiel, Germany
O'Sullivan, T., van Sinderen, D. & Fitzgerald, G. (1999). Structural and functional analysis of pCI65st, a 6.5 kb plasmid from Streptococcus thermophilus NDI-6. Microbiology 145, 127-134
Sambrook, J., Fritsch, E. F. & Maniatis, T. (1989). Molecular cloning: a laboratory manual, 2^{nd} ed. Cold Spring Harbor Laboratory, Cold Spring Harbor, N.Y.
Somkuti, G. A., Solaiman, D. K. Y. & Steinberg, D. H. (1998). Structural and functional properties of the hsp16.4-bearing plasmid pER341 in Streptococcus thermophilus. Plasmid 40, 61-72.
Gonzalez-Marquez, et al., (1997) Microbiology, vol. 143: 1587-1594.

All publications mentioned in the above specification are herein incorporated by reference. AU database sequences denoted by accession or gi numbers are likewise incorporated by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in food fermentation or related fields are intended to be within the scope of the following claims.

TABLE 1
Bacterial strains and bacteriophage

| Bacterial strains or phage | Relevant characteristics | Source or reference |
|---|---|---|
| S. thermophilus | | |
| S4 | pSt04, thermoresistant | BAFM^{a)} |
| S4-1 | plasmid-free S4 | this study |
| S4-1 (p99-17-2) | thermoresistant | this study |
| ER1 | pER1-2 (shsp), pER1-2 | BAFM |
| ER1-2 | pER1—1 removed | this study |
| S8 | pSt08 | BAFM |
| S8 (pSt04) | thermoresistant | this study |
| S8 (p99-17-2) | thermoresistant | this study |
| S11 | plasmid-free | Nestle, Vevey, |
| | | Switzerland |
| S11 (p99-17-2) | thermoresistant | this study |
| L. bulgaricus | | |
| 92063 | industrial strain | BAFM |
| HM | industrial strain | BAFM |
| L. lactis | | |
| MG1363 | plasmid-free L. lactis | Gasson, 1983 |
| | 712 | |
| MG1363 (p99-17-2) | thermoresistant | this study |
| IL1403 | plasmid-free | Chopin et al., 1984 |
| IL1403 (p99-17-2) | thermoresistant | this study |
| Bu2-60 | plasmid-free | BAFM |
| Bu2-60 (p99-17-2) | thermoresistant | this study |
| Bacteriophages s8 | small-isometric headed | BAFM |

^{a)}strain colletcion of the Institute of Microbiology, Bundesanstalt für Milchforschung, Kiel, Germany

TABLE 2
Changes of the pH-values of Yoghurt produced with S. thermophilus
S4 and L. bulgaricus 92063 at 42° and 50° C. during storage at 4° C.

| Fermentation | Time (days) | | | | | | |
|---|---|---|---|---|---|---|---|
| Temp. | 0 | 1 | 3 | 6 | 9 | 12 | 15 |
| 42° C. | 6.18 | 3.61 | 3.54 | 3.5 | 3.42 | 3.4 | 3.41 |
| 50° C. | 6.22 | 4.23 | 4.2 | 4.19 | 4.19 | 4.19 | 4.21 |

1. A method of preparing a milk product, the method comprising the steps of: combining milk with a lactic acid bacterium capable of expressing a small heat shock protein, and culturing the milk/bacterium mixture at a temperature higher than a regular fermentation temperature and/or at a salt concentration higher than a regular fermentation salt concentration wherein the milk product has milder taste characteristics when compared to a milk product produced with the corresponding lactic acid bacteria which do not express small heat shock proteins.

2. A method of preparing a milk product according to claim 1, wherein said milder taste characteristics product has a higher pH value when compared to the pH value of the milk product produced with the corresponding bacteria which do not express small heat shock proteins.

3. A method according to claim 1 wherein the pH of the product is above 4.0.

4. A method according to claim 1 wherein the bacterium comprises a mesophilic lactic acid bacterium and/or a thermophilic lactic acid bacterium.

5. A method according to claim 4, wherein the method comprises combining milk with a first and a second lactic acid bacterium, in which each of the first and/or second lactic acid bacteria is capable of expressing a small heat shock protein, and culturing the milk/bacterium mixture as specified.

6. A method according to claim 5, wherein each of said first lactic acid bacterium and said second lactic acid bacterium comprise a mesophilic bacterium.

7. A method according to claim 6, wherein the milk is combined with a starter culture comprising a first mesophilic lactic acid bacterium prior to the addition of a second mesophilic lactic acid bacterium.

8. A method according to claim 5, wherein each of said first lactic acid bacterium and said second lactic acid bacterium comprises a thermophilic bacterium.

9. A method according to claim 8, wherein the milk is combined with a starter culture comprising a first thermophilic lactic acid bacterium prior to the addition of a second thermophilic lactic acid bacterium.

10. A method according to claim 5, wherein the first lactic acid bacterium comprises a mesophilic lactic acid bacterium and the second lactic acid bacterium comprises a thermophilic lactic acid bacterium.

11. A method according to claim 10, wherein the milk is combined with a starter culture comprising a mesophilic lactic acid bacterium prior to the addition of a thermophilic lactic acid bacterium.

12. A method according to claim 11, wherein the milk is combined with a starter culture comprising a thermophilic lactic acid bacterium prior to the addition of a mesophilic lactic acid bacterium.

13. A method according to claim 7, wherein the starter culture comprising said first lactic bacterium and said second lactic acid bacterium are added to the milk at the same time.

14. A method according to claims 10, wherein the starter culture and the second culture are added at the same time.

15. A method according to claim 1, wherein said mesophilic bacterium is independently selected from a group consisting of: Lactococcus spp, Leuconostoc spp, Lactococcus lactis, Lactococcus lactis subspecies cremoris, and Lactacoccus lactis subspecies lactis.

16. A method according to claim 1, wherein said thermophilic bacterium is independently selected from a group consisting of: Streptococcus spp, B˜fidobacterium spp, Pediococcus spp, Lactobacillus spp, Streptococcus thermophilus, Lactobacillus debrueckii or Lactobacillus debrueckii subspecies bulgaricus.

17. A method according to claim 1, wherein said first thermophilic bacterium is Lactobacillus debrueckii or Lactobacillus debrueckif subspecies bulgaricus, and said second thermophilic bacterium is Streptococcus thermophilus.

18. A method according to claim 1, wherein said mesophilic lactic acid bacterium is capable of fermenting milk at up to 43° C.

19. A method according to claim 1, wherein said thermophilic lactic acid bacterium is capable of fermenting milk at up to 55° C.

20. A method according to claim 1, wherein the method comprises culturing the milk/bacterium mixture at lower pH range than the pH range for culturing milk with the corresponding bacteria which do not express small heat shock proteins.

21. A method according to claim 20, wherein the lactic acid bacteria remain viable at pH in the range of between 3.9 and 4.5.

22. A method according to claim 20, wherein at pH 3.9, at least 10⁶ of the lactic acid bacteria in the culture remain viable when stored for 15 days at 4° C.

23. A method according to claim 1, wherein the lactic acid bacterium comprises a recombinant lactic acid bacterium, which has been engineered to express a small heat shock protein at a higher level than a corresponding unmodified bacterium.

24-47. (canceled)

48. A method according to claim 1, wherein the lactic acid bacteria is a recombinant bacteria which has been engineered to express a small heat shock protein at a higher level than a corresponding lactic acid bacterium which do not express said small heat shock proteins.

49. A method according to claim 48, wherein the bacterium is capable of carrying out milk fermentation at temperatures and/or salt concentrations higher than regular fermentation temperatures and/or salt concentration.

50. A method of preparing a milk product, the method comprising the steps of: combining milk with a lactic acid bacterium capable of expressing a small heat shock protein, and culturing the milk/bacterium mixture at a temperature higher than a regular fermentation temperature and/or at a salt concentration higher than a regular fermentation salt concentration wherein the milk product is selected from a group consisting of acid curd cheese, hard cheese, semi-hard cheese like mozzarella, fresh cheese, quark, butter milk, Swiss type cheese or cottage cheese.

51. A milk product prepared by a method according to claim 1.

52. A composition comprising a recombinant lactic acid bacterium according to claim 48 together with milk or a milk product.


SEQUENCE LISTING (4 sequences)

SEQ ID NO: 1 - protein, 154 aa, Artificial Sequence
  Description of Artificial Sequence Synthetic peptide
MLNKIQQRNSDTYSVTPFNFFEDFRRNLFNDFFEDFSHNLFNDFKPNLIKTDIHETDNEYFVEAELPGIPKEDIQINYENGVLTISGQRQIDAATEDKKGKLIRNERSLTSVRRQYLLENVKEDEIKASYSDGILKVTLPKDSNKEIKTSIPIE

SEQ ID NO: 2 - protein, 142 aa, Artificial Sequence
  Description of Artificial Sequence Synthetic peptide
MLNKIQPRNSDTYSMTPFDFFEDFSRNLFNDFKSNLIKTDIHETDNEYLVEAELPGIPKENIQVTYEDGVLTISGQQQIDTVDEDKKGKLIRSERSSTSLRRQYLLENVKEDEIKASYSDGILKVTLPKDSNKEIKKSIPIE

SEQ ID NO: 3 - DNA, 462 bp, Artificial Sequence
  Description of Artificial Sequence Synthetic nucleotide sequence
ttcaattggaatagatgtttttatttctttattactatcttttggtaaggttactttaagaattccatctgaataagaagcctttatttcgtcttctttaacattttctaataaatattgacgtcggacacttgttaaactacgttcgttacgaatcaacttaccttttttatcttcggttgctgcatcaatttggcgttgaccactaattgttaatactccgttttcgtaattaatttgaatgtcttctttaggaataccaggaagttcagcctctacaaaatattcattatcagtttcatgaatatctgttttgattaaattgggcttaaaatcattgaataaattatgactaaagtcttcaaaaaaatcattgaataaattacgtctaaagtcttcaaaaaaattaaaaggtgtcacactatatgtgtctgaattacgttgttgaatcttattcaacat

SEQ ID NO: 4 - DNA, 426 bp, Artificial Sequence
  Description of Artificial Sequence Synthetic nucleotide sequence
ctcaattggaatagatttttttatttctttgttgctatcttttggcaaggttactttaagaattccatctgaataagaagcctttatttcgtcttctttaacattttctaataaatattgacgtcggagacttgttgaactacgttcgctacgaatcaactttccttttttatcttcgtctactgtatcaatttgttgttgcccactaattgttaatactccgtcttcgtaagtaacttgaatgttttctttaggaataccaggaagttcagcttctacaagatattcattatcagtttcatgaatatctgttttgattaaattggacttaaaatcattgaataaattacgactaaagtcttcaaaaaaatcaaaaggcgtcatactatatgtgtctgagttgcgtggttgaatcttatttaacat